(19) United States
(12) Patent Application Publication
(10) Pub. No.: US 2005/0032060 A1
(43) Pub. Date: Feb. 10, 2005

(54) Arrays comprising pre-labeled biological molecules and methods for making and using these arrays

(76) Inventors: Shishir Shah, Houston, TX (US); Mansoor Mohammed, Mission Viejo, CA (US)

Correspondence Address: MINTZ, LEVIN, COHN, FERRIS, GLOVSKY;AND POPEO, P.C., ONE FINANCIAL CENTER, BOSTON, MA, 02111, US

(21) Appl. No.: 10/488,492

(22) PCT Filed: Aug. 30, 2002
(86) PCT No.: PCT/US02/27743

Related U.S. Application Data
(60) Provisional application No. 60/316,347, filed on Aug. 31, 2001.

(51) Int. Cl. C12Q 1/68; C12M 1/34

(52) U.S. Cl. 435/6; 435/287.2

(57) ABSTRACT

The invention provides arrays comprising a plurality of biological molecules, wherein each biological molecule is immobilized to a discrete and known spot on a substrate surface to form an array of biological molecules, and each biological molecule comprises a detectable label. The invention provides a multiplexed system for performing comparative genomic hybridization (CGH) using an array of the invention. The invention also provides methods of making and using these arrays, including a method of comparing copy numbers of unique nucleic acid sequences in a sample.

### TECHNICAL FIELD

This invention relates to molecular biology, genetic diagnostics and array, or “biochip,” technology. In particular, the invention provides novel arrays, particularly nucleic acid arrays, comprising a plurality of biological molecules, wherein each biological molecule is immobilized to a discrete and known spot on a substrate surface to form an array of biological molecules, and each biological molecule comprises a detectable label.

### BACKGROUND

Genomic DNA microarray based comparative genomic hybridization (CGH) has the potential to solve many of the limitations of traditional CGH method, which relies on comparative hybridization on individual metaphase chromosomes. In metaphase CGH, multi-megabase fragments of different samples of genomic DNA (e.g., known normal versus test, e.g., a possible tumor) are labeled and hybridized to a fixed chromosome (see, e.g., Breen (1999) J. Med. Genetics 36:511-517; Rice (2000) Pediatric Hematol. Oncol. 17:141-147). Signal differences between known and test samples are detected and measured. In this way, missing, amplified, or unique sequences in the test sample, as compared to “normal,” can be detected by the fluorescence ratio of normal control to test genomic DNA. In metaphase CGH, the target sites (on the fixed chromosome) are saturated by an excess amount of soluble, labeled genomic DNA.

In contrast to metaphase CGH, where the immobilized genomic DNA is a metaphase spread, in array-based CGH method the immobilized nucleic acids are arranged as an array, on, e.g., a biochip or a microarray platform. Another difference is that in array-based CGH the immobilized genomic DNA is in molar excess as compared to the copy number of labeled (test and control) genomic nucleic acid. Under such conditions, suppression of repetitive genomic sequences and cross hybridization on the immobilized DNA is very helpful for reliable detection and quantitation of copy number differences between normal control and test samples. However, when traditional protocols are used such suppression is less than optimal. Furthermore, genomic DNA is a promiscuous mix containing more than 30% repetitive sequences and a further unknown proportion of closely related sequences. These sequences can cross-hybridize when traditional protocols are used to prepare test and sample DNA for hybridization to the array.

### SUMMARY

The invention provides an array comprising a plurality of biological molecules, wherein each biological molecule is immobilized to a discrete and known spot on a substrate surface to form an array of biological molecules, and each biological molecule comprises a detectable label.

In alternative aspects, the biological molecule comprises a nucleic acid, e.g., an oligonucleotide, a lipid, a polysaccharide, a polypeptide (e.g., a peptide), or an analog or a mimetic thereof, or a combination thereof. The nucleic acid can comprise a DNA (e.g., a genomic DNA or a cDNA), an RNA (e.g., an mRNA, rRNA, and the like) or an analog or a mimetic thereof or a combination thereof. The nucleic acid can further comprise a telomeric structure or a chromatin structure. Analogs and mimetics can include small molecules, as discussed below.

In one aspect, the biological molecule, e.g., the biological molecule, e.g., a nucleic acid, is derived from a mammal, e.g., a human. The nucleic acids can be genomic DNA or can be derived from a genomic DNA. The nucleic acids can comprise cloned nucleic acid segments, such as cloned genomic nucleic acid segments.

In one aspect, the cloned genomic nucleic acid segments are free of deletions, as compared to a corresponding wild type genomic nucleic acid segment. The cloned genomic nucleic acid segment can be free of additions, as compared to a corresponding wild type genomic nucleic acid segment. Alternatively, the cloned genomic nucleic acid segment can be free of additions and deletions, as compared to a corresponding wild type genomic nucleic acid segment.

In alternative aspects, the cloned genomic nucleic acid segment comprises a gene, a cDNA or a DNA sequence corresponding to or complementary to an RNA message. The message can comprise sequence encoding a polypeptide.

In some aspects of the arrays of the invention, each spot (on a substrate surface) consists of a plurality of nucleic acids comprising a single cloned genomic nucleic acid segment. In one aspect, the cloned genomic nucleic acid segments in a first spot are non-overlapping in sequence compared to the cloned genomic nucleic acid segments in a second spot. Alternatively, the cloned genomic nucleic acid segments in a spot are non-overlapping in sequence compared to the cloned genomic nucleic acid segments all of other genomic nucleic acid-comprising spots on the array.

In alternative aspects, each cloned genomic nucleic acid segment is spotted in duplicate or triplicate on the array. The cloned genomic nucleic acid segments together can comprise a known segment of a genome. The known segment of a genome can comprise a substantially complete chromosome, such as a mammalian chromosome, e.g., a human chromosome. The known segment of a genome can comprise a substantially complete genome, such as a mammalian genome, e.g., a human genome.

In alternative aspects, the cloned nucleic acid segment is cloned in a construct comprising an artificial chromosome, such as a bacterial artificial chromosome (BAC), a human artificial chromosome (HAC), a yeast artificial chromosome (YAC), a transformation-competent artificial chromosome (TAC) or a bacteriophage P1-derived artificial chromosome (PAC). The cloned nucleic acid segment can be cloned in a construct comprising a vector selected from the group consisting of a cosmid vector, a plasmid vector and a viral vector.

In alternative aspects, the cloned nucleic acid segment is between about 5 kilobases (0.05 megabase) to about 1000 kilobases (10 megabases) in length, between about 50 kilobases (0.5 megabase) to about 500 kilobases (5 megabases) in length, between about 100 kilobases (1 megabase) to about 400 kilobases (4 megabases) in length, and about 300 kilobases (3 megabases) in length.

In alternative aspects, the detectable label comprises a fluorescent label, such as a Cy5™ or equivalent, a Cy3™ or equivalents; and, a rhodamine, a fluorescein or an aryl-substituted 4,4-difluoro-4-bora-3a,4a-diaza-s-indacene dye or equivalents.

In alternative aspects, the biological molecule is covalently and non-covalently bound to the detectable label. As described herein, labels also can be incorporated into the biological molecule, as with labeled nucleosides incorporated into a nucleic acid by, e.g., nick translation, random primer extension, amplification with degenerate primers (by, e.g., PCR), and the like.

In one aspect, all of the array-immobilized biological molecules comprise the same detectable label. However, a biological molecule also can comprise two or more labels, or, two or more labels can be associated with the array-immobilized biological molecules.

In one aspect, about 50%, about 60%, about 70%, about 80%, about 90%, about 95%, about 99%, or 100% of the array-immobilized biological molecules comprise at least one detectable label. For example, in one aspect, some biological molecules on a “spot” can be unlabeled, i.e., it is not necessary that all of the biological molecules on a “spot” be labeled. In another aspect, an array can have “spots” of biological molecules that are not labeled, or, are differently labeled as compared to the same biological molecules on other spots on the array.

In alternative aspects, the biological molecule is covalently and non- covalently bound to the substrate surface. In one aspect, the biological molecule is covalently bound to a compound having the general formula: R₁—X—R₂, wherein R₁ is a cyclic ether, an aldehyde, or a chloromethylphenyl moiety; X is a moiety chemically suitable for linking the R₁ moiety to the R₂ moiety, and the R₂ moiety has the general formula 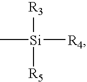
wherein R₃, R₄ and R₅ comprise identical or different alkoxy group or chloro groups. The cyclic ether can be an epoxide group. The epoxide group can be an epoxycyclohexyl group or epoxycyclopentyl group. The R₂ moiety can comprise an alkoxysilane group. The R₂ moiety can be selected from the group consisting of a methoxyethoxy group, a —OCH₂CH₂OCH₃ group, a chlorohalide group and a propoxy group. The R₁—X—R₂ compound can be selected from the group consisting of (3-glycidoxypropyl) methyldiethoxysilane, (3-glycidoxypropyl) methyldiisopropenoxy-silane; 2-(3,4-epoxycyclohexyl)ethyltrimethoxysilane and (3-glycidoxypropyl) dimethylethoxysilane.

In one aspect, the biological molecule is covalently bound to a compound having the general formula: R₁—X—R₂, wherein R₁ is an amino group, R₂ is an alkoxysilane group or a chlorohalide group; and X is a moiety chemically suitable for linking the R₁ group and the R₂ group.

In one aspect, the biological molecule is covalently bound to a compound having the general formula
R₁—X—Si(OR₂)ₘ(Cl)ₙ(R)ₖ,

wherein m+k is the integer 3, and n can be 0 if m is greater than 0, or n+k is the integer 3 and m can be 0 if n is greater than 0; X is an inert linker; R₁ comprises a group reactive toward the biological molecule; R is an alkyl group; and, R₂ is an alkyl group.

The invention provides an array comprising a plurality of bacterial artificial chromosomes (BACs), wherein each bacterial artificial chromosome is immobilized to a discrete and known spot on a substrate surface to form an array of bacterial artificial chromosomes (BACs), and each bacterial artificial chromosome comprises a detectable label. The array can comprise a SpectralChip™ Mouse BAC Array. The array can comprise a SpectralChip™ Human BAC Array.

The invention provides a method of making an array, wherein the array comprises a plurality of genomic nucleic acids covalently labeled with a compound comprising a fluorescent label, comprising the following steps (a) providing plurality of genomic nucleic acid segments, wherein the segments have lengths averaging between about 0.5 megabase and about 5 megabases or are fragmented or digested to have lengths averaging between about 0.5 megabase and about 5 megabases; (b) labeling the genomic nucleic acid segments of step (a) with a fluorescent label; and, (c) immobilizing the random prime labeled nucleic acids to discrete and known spots on a substrate surface to form an array of nucleic acids. In alternative aspects, the cloned nucleic acid segment is cloned in a construct comprising an artificial chromosome, such as a bacterial artificial chromosome (BAC), a yeast artificial chromosome (YAC), a human artificial chromosome (HAC), a bacteriophage P1-derived artificial chromosome (PAC), or, a cosmid or a plasmid.

In one aspect, the detectable label comprises a fluorescent label. The fluorescent label comprises Cy5™ or equivalent, a Cy3™ or equivalent, or, a rhodamine, a fluorescein or an aryl-substituted 4,4-difluoro-4-bora-3a,4a-diaza-s-indacene dye or equivalents.

In one aspect, the labeling of the nucleic acid segments (e.g., genomic DNA) comprises random prime labeling, nick translation labeling, amplification with degenerate primers (by, e.g., PCR), and the like.

The invention provides a method of performing comparative genomic hybridization (CGH) using an array comprising a plurality of genomic nucleic acids covalently labeled with a compound comprising a detectable label, comprising the following steps: (a) providing an array comprising a plurality of cloned genomic nucleic acid segments, wherein each genomic nucleic acid segment is immobilized to a discrete and known spot on a substrate surface to form an array, and the genomic nucleic acid segments comprise a first detectable label; (b) providing a sample comprising a plurality of genomic nucleic acid segments, wherein the sample genomic nucleic acid segments are labeled with at least a second detectable label; (c) contacting the sample of step (b) with the array of step (a) under conditions wherein the nucleic acid in the sample can specifically hybridize to the genomic nucleic acid segments of step (a); and, (d) measuring the amount of first and second fluorescent label on each spot, thereby performing comparative genomic hybridization. In one aspect, the cloned nucleic acid segment is cloned in a construct comprising an artificial chromosome, such as a bacterial artificial chromosome (BAC), YAC, HAC, P1, and the like. In one aspect, the detectable label comprises a fluorescent label, such as a Cy5™ or equivalent, a Cy3™ or equivalent, or, a rhodamine, a fluorescein or an aryl-substituted 4,4-difluoro-4bora-3a,4a-diaza-s-indacene dye or equivalents.

The invention provides a method of comparing copy numbers of unique nucleic acid sequences in a first sample comprising nucleic acid of a cell or cell population relative to copy numbers of substantially identical sequences in at least a second sample comprising nucleic acid of at least a second cell or cell population, said method comprising the steps of: (a) providing an array comprising the nucleic acid of a first sample, wherein the nucleic acid is immobilized to discrete and known spots on a substrate surface to form an array, and the nucleic acids of a first sample comprise a first detectable label; (b) providing a second sample comprising nucleic acid labeled with a second detectable label; (c) contacting the sample of step (b) with the array of step (a) under conditions wherein the nucleic acid in the sample can specifically hybridize to the immobilized nucleic acid of step (a); and, (d) comparing the intensities of the signals from the labeled sample nucleic acids hybridized to the immobilized nucleic acids of step (a), thereby comparing copy numbers of unique nucleic acid sequences in a first sample compared to the second sample.

The invention provides a multiplexed system for performing comparative genomic hybridization (CGH) using an array comprising: (a) an array comprising a plurality of biological molecules, wherein each biological molecule is immobilized to a discrete and known spot on a substrate surface to form an array of biological molecules, and each biological molecule comprises a first detectable label; (b) a device for detecting the array-immobilized detectable label and at least one second detectable label, wherein the device can measure which detectable labels are on which spots on the substrate surface. In one aspect, the device of step (b) comprises a charge-coupled device (CCD).

The details of one or more embodiments of the invention are set forth in the accompanying drawings and the description below. Other features, objects, and advantages of the invention will be apparent from the description and drawings, and from the claims.

All publications, patents, patent applications, GenBank sequences and ATCC deposits cited herein are hereby expressly incorporated by reference for all purposes.

### BRIEF DESCRIPTION OF THE DRAWINGS

The following drawings are illustrative of embodiments of the invention and are not meant to limit the scope of the invention as encompassed by the claims.

FIG. 1 is a schematic illustration of an exemplary humidified hybridization chamber and an unbalanced humidity hybridization format of the invention.

Like reference symbols in the various drawings indicate like elements.

### DETAILED DESCRIPTION

The invention provides novel methods and compositions for array-based nucleic acid hybridizations. New methods and compositions are provided for generating a molecular profile of genomic DNA by hybridization of a target nucleic acid derived from genomic DNA to an immobilized nucleic acid probe, e.g., as in an “array-based comparative genomic hybridization (CGH).”

In one aspect, the invention provides an array comprising a plurality of biological molecules, such as genomic DNA, wherein each biological molecule is immobilized to a discrete and known spot on a substrate surface to form an array of biological molecules, and each immobilized biological molecule comprises a detectable label. One or more detectable moieties can be used to label the molecules immobilized on the array. In practicing the methods of the invention, in one aspect, the sample of biological molecules is labeled with one or more detectable labels that are different from the one or more detectable moieties used to label the molecules immobilized on the array.

In one aspect, the invention provides a method for generating a molecular profile of one or more genomes, or a defined portion of a genome, e.g., a chromosome or part of a chromosome, by hybridization of target nucleic acid derived from a genomic DNA to an immobilized nucleic acid probe(s), e.g., in the form of an array. The method comprises contacting the (labeled) immobilized nucleic acid segment (e.g., cloned DNA) with a sample of target nucleic acid comprising fragments of genomic nucleic acid labeled with a detectable moiety.

### Definitions

Unless defined otherwise, all technical and scientific terms used herein have the meaning commonly understood by a person skilled in the art to which this invention belongs. As used herein, the following terms have the meanings ascribed to them unless specified otherwise.

The term “aryl-substituted 4,4-difluoro-4-bora-3a,4a-diaza-s-indacene dye” as used herein includes all “boron dipyrromethene difluoride fluorophore” or “BODIPY” dyes and “dipyrrometheneboron difluoride dyes” (see, e.g., U.S. Pat. No. 4,774,339), or equivalents, are a class of fluorescent dyes commonly used to label nucleic acids for their detection when used in hybridization reactions; see, e.g., Chen (2000) J. Org. Chem. 65:2900-2906: Chen (2000) J. Biochem. Biophys. Methods 42:137-151. See also U.S. Pat. Nos. 6,060,324; 5,994,063; 5,614,386; 5,248,782; 5,227,487; 5,187,288.

The terms “cyanine 5” or “Cy5™” and “cyanine 3” or “Cy3™” refer to fluorescent cyanine dyes produced by Amersham Pharmacia Biotech (Piscataway, N.J.) (Amersham Life Sciences, Arlington Heights, Ill.), as described in detail, below, or equivalents. See U.S. Pat. Nos. 6,027,709; 5,714,386; 5,268,486; 5,151,507; 5,047,519. These dyes are typically incorporated into nucleic acids in the form of 5-amino-propargyl-2′-deoxycytidine 5′-triphosphate coupled to Cy5™ or Cy3™.

The term “fluorescent dye” as used herein includes all known fluors, including rhodamine dyes (e.g., tetramethylrhodamine, dibenzorhodamine, see, e.g., U.S. Pat. No. 6,051,719); fluorescein dyes; “BODIPY” dyes and equivalents (e.g., dipyrrometheneboron difluoride dyes, see, e.g., U.S. Pat. No. 5,274,113); derivatives of 1-[isoindolyl]methylene-isoindole (see, e.g., U.S. Pat. No. 5,433,896); and all equivalents. See also U.S. Pat. Nos. 6,028,190; 5,188,934.

The terms “hybridizing specifically to” and “specific hybridization” and “selectively hybridize to,” as used herein refer to the binding, duplexing, or hybridizing of a nucleic acid molecule preferentially to a particular nucleotide sequence under stringent conditions. The term “stringent conditions” refers to conditions under which a probe will hybridize preferentially to its target subsequence, and to a lesser extent to, or not at all to, other sequences. A “stringent hybridization” and “stringent hybridization wash conditions” in the context of nucleic acid hybridization (e.g., as in array, Southern or Northern hybridizations) are sequence dependent, and are different under different environmental parameters. Stringent hybridization conditions that can be used to identify nucleic acids can include, e.g., hybridization in a buffer comprising 50% formamide, 5×SSC, and 1% SDS at 42° C., or hybridization in a buffer comprising 5×SSC and 1% SDS at 65° C., both with a wash of 0.2×SSC and 0.1% SDS at 65° C. Exemplary stringent hybridization conditions can also include a hybridization in a buffer of 40% formamide, 1 M NaCl, and 1% SDS at 37° C., and a wash in 1×SSC at 45° C. Those of ordinary skill will readily recognize that alternative but comparable hybridization and wash conditions can be utilized to provide conditions of similar stringency. However, the selection of a hybridization format is not critical, as is known in the art, it is the stringency of the wash conditions that set forth the conditions which determine whether a soluble, sample nucleic acid will specifically hybridize to an immobilized nucleic acid. Wash conditions used to identify nucleic acids include, e.g.: a salt concentration of about 0.02 molar at pH 7 and a temperature of at least about 50° C. or about 55° C. to about 60° C.; or, a salt concentration of about 0.15 M NaCl at 72° C. for about 15 minutes; or, a salt concentration of about 0.2×SSC at a temperature of at least about 50° C. or about 55° C. to about 60° C. for about 15 to about 20 minutes; or, the hybridization complex is washed twice with a solution with a salt concentration of about 2×SSC containing 0.1% SDS at room temperature for 15 minutes and then washed twice by 0.1×SSC containing 0.1% SDS at 68° C. for 15 minutes; or, equivalent conditions. Stringent conditions for washing can also be, e.g., 0.2×SSC/0.1% SDS at 42° C. In instances wherein the nucleic acid molecules are deoxyoligonucleotides (“oligos”), stringent conditions can include washing in 6×SSC/0.05% sodium pyrophosphate at 37° C. (for 14-base oligos), 48° C. (for 17-base oligos), 55° C. (for 20-base oligos), and 60° C. (for 23-base oligos). See Sambrook, Ausubel, or Tijssen (cited below) for detailed descriptions of equivalent hybridization and wash conditions and for reagents and buffers, e.g., SSC buffers and equivalent reagents and conditions.

The phrase “labeled with a detectable composition” or “labeled with a detectable moiety” as used herein refers to a biological molecule, e.g., a polypeptide or a nucleic acid, comprising a detectable composition, i.e., a label, as described in detail, below. The label can also be another biological molecule, as a nucleic acid, e.g., a nucleic acid in the form of a stem-loop structure as a “molecular beacon,” as described below. This includes incorporation of labeled bases (or, bases which can bind to a detectable label) into the nucleic acid by, e.g., nick translation, random primer extension, amplification with degenerate primers, and the like. The label can be detectable by any means, e.g., visual, spectroscopic, photochemical, biochemical, immunochemical, physical or chemical means. The invention provides arrays comprising immobilized biological molecules, e.g., nucleic acids, comprising detectable labels.

The term “a molecular profile of genomic DNA” means detection of regions of amplification, deletions and/or unique sequences in a test sample of nucleic acid representing a genomic DNA as compared to a control (e.g., “normal”) sample of DNA. “Genomic DNA” or “genomic nucleic acid” includes sense and complementary strands, cloned and amplified copies, synthetic copies, or otherwise reproduced sequences of a genomic nucleic acid.

The term “nucleic acid” as used herein refers to a deoxyribonucleotide or ribonucleotide in either single- or double-stranded form. The term encompasses nucleic acids containing known analogues of natural nucleotides. The term also encompasses nucleic-acid-like structures with synthetic backbones. DNA backbone analogues provided by the invention include phosphodiester, phosphorothioate, phosphorodithioate, methylphosphonate, phosphoramidate, alkyl phosphotriester, sulfamate, 3′-thioacetal, methylene(methylimino), 3′-N-carbamate, morpholino carbamate, and peptide nucleic acids (PNAs); see Oligonucleotides and Analogues, a Practical Approach, edited by F. Eckstein, IRL Press at Oxford University Press (1991); Antisense Strategies, Annals of the New York Academy of Sciences, Volume 600, Eds. Baserga and Denhardt (NYAS 1992); Milligan (1993) J. Med. Chem. 36:1923-1937; Antisense Research and Applications (1993, CRC Press). PNAs contain non-ionic backbones, such as N-(2-aminoethyl) glycine units. Phosphorothioate linkages are described, e.g., by U.S. Pat. Nos. 6,031,092; 6,001,982; 5,684,148; see also, WO 97/03211; WO 96/39154; Mata (1997) Toxicol. Appl. Pharmacol. 144:189-197. Other synthetic backbones encompassed by the term include methyl-phosphonate linkages or alternating methylphosphonate and phosphodiester linkages (see, e.g., U.S. Pat. No. 5,962,674; Strauss-Soukup (1997) Biochemistry 36:8692-8698), and benzylphosphonate linkages (see, e.g., U.S. Pat. No. 5,532,226; Samstag (1996) Antisense Nucleic Acid Drug Dev 6:153-156). The term nucleic acid is used interchangeably with gene, DNA, RNA, cDNA, mRNA, oligonucleotide primer, probe and amplification product.

The terms “polypeptide,” “protein,” and “peptide” include compositions of the invention that also include “analogs,” or “conservative variants” and “mimetics” or “peptidomimetics” with structures and activity that substantially correspond to the polypeptide from which the variant was derived, as discussed in detail, below.

The term “small molecule” means any synthetic small molecule, such as an organic molecule or a synthetic molecule, such as those generated by combinatorial chemistry methodologies. These small molecules can be synthesized using a variety of procedures and methodologies, which are well described in the scientific and patent literature, e.g., Organic Syntheses Collective Volumes, Gilman et al. (Eds) John Wiley & Sons, Inc., NY; Venuti (1989) Pharm Res. 6:867-873. Synthesis of small molecules, as with all other procedures associated with this invention, can be practiced in conjunction with any method or protocol known in the art. For example, preparation and screening of combinatorial chemical libraries are well known, see, e.g., U.S. Pat. Nos. 6,096,496; 6,075,166; 6,054,047; 6,004,617; 5,985,356; 5,980,839; 5,917,185; 5,767,238.

The terms “array” or “microarray” or “DNA array” or “nucleic acid array” or “biochip” as used herein is a plurality of target elements, each target element comprising a defined amount of one or more biological molecules, e.g., nucleic acids, immobilized a solid surface for hybridization to sample biological molecules, e.g., nucleic acids.

The term “sample of nucleic acid targets” or “sample of nucleic acid” as used herein refers to a sample comprising DNA or RNA, or nucleic acid representative of DNA or RNA isolated from a natural source, in a form suitable for hybridization (e.g., as a soluble aqueous solution) to another nucleic acid or polypeptide or combination thereof (e.g., immobilized probes). The nucleic acid may be isolated, cloned or amplified; it may be, e.g., genomic DNA, mRNA, or cDNA from substantially an entire genome, substantially all or part of a particular chromosome, or selected sequences (e.g. particular promoters, genes, amplification or restriction fragments, cDNA, etc.). The nucleic acid sample may be extracted from particular cells or tissues. The cell or tissue sample from which the nucleic acid sample is prepared is typically taken from a patient suspected of having a genetic defect or a genetically-linked pathology or condition, e.g., a cancer, associated with genomic nucleic acid base substitutions, amplifications, deletions and/or translocations. Methods of isolating cell and tissue samples are well known to those of skill in the art and include, but are not limited to, aspirations, tissue sections, needle biopsies, and the like. Frequently the sample will be a “clinical sample” which is a sample derived from a patient, including sections of tissues such as frozen sections or paraffin sections taken for histological purposes. The sample can also be derived from supernatants (of cells) or the cells themselves from cell cultures, cells from tissue culture and other media in which it may be desirable to detect chromosomal abnormalities or determine amplicon copy number. In some cases, the nucleic acids may be amplified using standard techniques such as PCR, prior to the hybridization. The immobilized biological molecule is labeled, as described herein. The probe an be produced from and collectively can be representative of a source of nucleic acids from one or more particular (pre-selected) portions of, e.g., a collection of polymerase chain reaction (PCR) amplification products, substantially an entire chromosome or a chromosome fragment, or substantially an entire genome, e.g., as a collection of clones, e.g., BACs, PACs, YACs, and the like (see below). The probe or genomic nucleic acid sample may be processed in some manner, e.g., by blocking or removal of repetitive nucleic acids or by enrichment with selected nucleic acids.

As used herein, the terms “computer” and “processor” are used in their broadest general contexts and incorporate all such devices. The methods of the invention can be practiced using any computer/processor and in conjunction with any known software or methodology. For example, a computer/processor can be a conventional general-purpose digital computer, e.g., a personal “workstation” computer, including conventional elements such as microprocessor and data transfer bus. The computer/processor can further include any form of memory elements, such as dynamic random access memory, flash memory or the like, or mass storage such as magnetic disc optional storage.

Generating and Manipulating Nucleic Acids

The invention provides nucleic acid arrays and methods for performing nucleic acid hybridization reactions. As described herein, the target nucleic acid for analysis and the immobilized nucleic acid on the array can be representative of genomic DNA, including defined parts of, or entire, chromosomes, or entire genomes. In various aspects of the invention, the arrays and methods of the invention are used in comparative genomic hybridization (CGH) reactions; see, e.g., U.S. Pat. Nos. 5,830,645; 5,976,790. These reactions compare the genetic composition of test versus controls samples; e.g., whether a test sample of genomic DNA (e.g., from a cell suspected of having a genetic defect) has amplified or deleted or mutated segments, as compared to a “negative” control, e.g., “normal” wild type genotype, or “positive” control, e.g., known cancer cell or cell with a known defect, e.g., a translocation or amplification or the like.

In other aspects, the test sample comprises fragments of nucleic acid representative of defined parts of a chromosome or genome, or the entire genome. The test sample also can be labeled, e.g., with a detectable moiety, e.g., a fluorescent dye. The test sample nucleic acid can labeled with a fluor and the control (e.g., “normal”) sample is labeled with a second dye (e.g., Cy3™ and Cy5™). In one aspect, the sample nucleic acid is labeled with different detectable moieties, e.g., different fluorescent dyes, than those used to label the immobilized nucleic acids.

Test and control samples are both applied to the immobilized probes (e.g., on the array) and, after hybridization and washing, the location (e.g., spots on the array) and amount of each dye are read. The immobilized nucleic acid can be representative of any part of or all of a chromosome or genome. If immobilized to an array, this nucleic acid can be in the form of cloned DNA, e.g., YACs, BACs, PACs, and the like, as described herein. As is typical of array technology, each “spot” on the array has a known sequence, e.g., a known segment of genome or other sequence. The invention can be practiced in conjunction with any method or protocol or device known in the art, which are well described in the scientific and patent literature.

General Techniques

The nucleic acids used to practice this invention, whether RNA, cDNA, genomic DNA, vectors, viruses or hybrids thereof, may be isolated from a variety of sources, genetically engineered, amplified, and/or expressed/generated recombinantly. Any recombinant expression system can be used, including, in addition to bacterial cells, e.g., mammalian, yeast, insect or plant cell expression systems.

Alternatively, these nucleic acids can be synthesized in vitro by well-known chemical synthesis techniques, as described in, e.g., Carruthers (1982) Cold Spring Harbor Symp. Quant. Biol. 47:411-418; Adams (1983) J. Am. Chem. Soc. 105:661; Belousov (1997) Nucleic Acids Res. 25:3440-3444; Frenkel (1995) Free Radic. Biol. Med. 19:373-380; Blommers (1994) Biochemistry 33:7886-7896; Narang (1979) Meth. Enzymol. 68:90; Brown (1979) Meth. Enzymol. 68:109; Beaucage (1981) Tetra. Lett. 22:1859; U.S. Pat. No. 4,458,066. Double stranded DNA fragments may then be obtained either by synthesizing the complementary strand and annealing the strands together under appropriate conditions, or by adding the complementary strand using DNA polymerase with a primer sequence.

Techniques for the manipulation of nucleic acids, such as, e.g., subcloning, labeling probes (e.g., random-primer labeling using Klenow polymerase, nick translation, amplification), sequencing, hybridization and the like are well described in the scientific and patent literature, see, e.g., Sambrook, ed., MOLECULAR CLONING: A LABORATORY MANUAL (2ND ED.), Vols. 1-3, Cold Spring Harbor Laboratory, (1989); CURRENT PROTOCOLS IN MOLECULAR BIOLOGY, Ausubel, ed. John Wiley & Sons, Inc., New York (1997); LABORATORY TECHNIQUES IN BIOCHEMISTRY AND MOLECULAR BIOLOGY: HYBRIDIZATION WITH NUCLEIC ACID PROBES, Part I. Theory and Nucleic Acid Preparation, Tijssen, ed. Elsevier, N.Y. (1993).

Another useful means of obtaining and manipulating nucleic acids used in the compositions and methods of the invention is to clone from genomic samples, and, if necessary, screen and re-clone inserts isolated (or amplified) from, e.g., genomic clones or cDNA clones or other sources of complete genomic DNA. Thus, forms of genomic nucleic acid used in the methods and compositions of the invention (including arrays and test samples) include genomic or cDNA libraries contained in, or comprised entirely of, e.g., mammalian artificial chromosomes (see, e.g., Ascenzioni (1997) Cancer Lett. 118:135-142; U.S. Pat. Nos. 5,721,118; 6,025,155) (including human artificial chromosomes, see, e.g., Warburton (1997) Nature 386:553-555; Roush (1997) Science 276:38-39; Rosenfeld (1997) Nat. Genet. 15:333-335); yeast artificial chromosomes (YAC); bacterial artificial chromosomes (BAC); P1 artificial chromosomes (see, e.g., Woon (1998) Genomics 50:306-316; Boren (1996) Genome Res. 6:1123-1130); PACs (a bacteriophage P1-derived vector, see, e.g., Ioannou (1994) Nature Genet. 6:84-89; Reid (1997) Genomics 43:366-375; Nothwang (1997) Genomics 41:370-378; Kern (1997) Biotechniques 23:120-124); cosmids, plasmids or cDNAs. BACs are vectors that can contain 120 Kb or greater inserts. BACs are based on the E. coli F factor plasmid system and simple to manipulate and purify in microgram quantities. Because BAC plasmids are kept at one to two copies per cell, the problems of rearrangement observed with YACs, which can also be employed in the present methods, are eliminated; see, e.g., Asakawa (1997) Gene 69-79; Cao (1999) Genome Res. 9:763-774. BAC vectors can include marker genes, such as, e.g., luciferase and green fluorescent protein genes (see, e.g., Baker (1997) Nucleic Acids Res 25:1950-1956). YACS can also be used and contain inserts ranging in size from 80 to 700 kb, see, e.g., Tucker (1997) Gene 199:25-30; Adam (1997) Plant J. 11:1349-1358; Zeschnigk (1999) Nucleic Acids Res. 27:21. P1 is a bacteriophage that infects E. coli that can contain 75-100 Kb DNA inserts (see, e.g., Mejia (1997) Genome Res 7:179-186; Ioannou (1994) Nat Genet 6:84-89), and are screened in much the same way as lambda libraries. See also Ashworth (1995) Analytical Biochem. 224:564-571; Gingrich (1996) Genomics 32:65-74. Sequences, inserts, clones, vectors and the like can be isolated from natural sources, obtained from such sources as ATCC or GenBank libraries or commercial sources, or prepared by synthetic or recombinant methods.

Amplification of Nucleic Acids

Amplification using oligonucleotide primers can be used to generate nucleic acids used in the compositions and methods of the invention, to incorporate label into immobilized or sample nucleic acids, to detect or measure levels of test or control samples hybridized to an array, and the like. Amplification, typically with degenerate primers, is also useful for incorporating detectable probes (e.g., Cy5™- or Cy3™-cytosine conjugates) into nucleic acids representative of test or control genomic DNA to be used to hybridize to immobilized genomic DNA. The skilled artisan can select and design suitable oligonucleotide amplification primers. Amplification methods are also well known in the art, and include, e.g., polymerase chain reaction, PCR (PCR PROTOCOLS, A GUIDE TO METHODS AND APPLICATIONS, ed. Innis, Academic Press, N.Y. (1990) and PCR STRATEGIES (1995), ed. Innis, Academic Press, Inc., N.Y., ligase chain reaction (LCR) (see, e.g., Wu (1989) Genomics 4:560; Landegren (1988) Science 241:1077; Barringer (1990) Gene 89:117); transcription amplification (see, e.g., Kwoh (1989) Proc. Natl. Acad. Sci. USA 86:1173); and, self-sustained sequence replication (see, e.g., Guatelli (1990) Proc. Natl. Acad. Sci. USA 87:1874); Q Beta replicase amplification (see, e.g., Smith (1997) J. Clin. Microbiol. 35:1477-1491), automated Q-beta replicase amplification assay (see, e.g., Burg (1996) Mol. Cell. Probes 10:257-271) and other RNA polymerase mediated techniques (e.g., NASBA, Cangene, Mississauga, Ontario); see also Berger (1987) Methods Enzymol. 152:307-316; Sambrook; Ausubel; U.S. Pat. Nos. 4,683,195 and 4,683,202; Sooknanan (1995) Biotechnology 13:563-564. See, e.g., U.S. Pat. No. 6,063,571, describing use of polyamide-nucleic acid derivatives (PNAs) in amplification primers.

Hybridizing Nucleic Acids

In practicing the methods of the invention and using the compositions of the invention, test and control samples of nucleic acid are hybridized to immobilized probe nucleic acid, e.g., on arrays. In one aspect, the hybridization and/or wash conditions are carried out under moderate to stringent conditions. An extensive guide to the hybridization of nucleic acids is found in, e.g., Sambrook Ausubel, Tijssen. Generally, highly stringent hybridization and wash conditions are selected to be about 5° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. Very stringent conditions are selected to be equal to the Tₘ for a particular probe. An example of stringent hybridization conditions for hybridization of complementary nucleic acids which have more than 100 complementary residues on an array or a filter in a Southern or northern blot is 42° C. using standard hybridization solutions (see, e.g., Sambrook), with the hybridization being carried out overnight. An example of highly stringent wash conditions is 0.15 M NaCl at 72° C. for about 15 minutes. An example of stringent wash conditions is a 0.2×SSC wash at 65° C. for 15 minutes (see, e.g., Sambrook). Often, a high stringency wash is preceded by a medium or low stringency wash to remove background probe signal. An example medium stringency wash for a duplex of, e.g., more than 100 nucleotides, is 1×SSC at 45° C. for 15 minutes. An example of a low stringency wash for a duplex of, e.g., more than 100 nucleotides, is 4× to 6×SSC at 40° C. for 15 minutes.

In alternative aspects of the compositions and methods of the invention, e.g., in practicing comparative hybridization (CGH) with the arrays of the invention, the fluorescent dyes Cy3™ and Cy5™ are used to differentially label nucleic acid fragments from two samples, e.g., the array-immobilized nucleic acid versus the sample nucleic acid, or, nucleic acid generated from a control versus a test cell or tissue. Many commercial instruments are designed to accommodate to detection of these two dyes. To increase the stability of Cy5™, or fluors or other oxidation-sensitive compounds, antioxidants and free radical scavengers can be used in hybridization mixes, the hybridization and/or the wash solutions. Thus, Cy5™ signals are dramatically increased and longer hybridization times are possible. See co-pending U.S. patent application Ser. No. 09/839,658, filed Apr. 19, 2001.

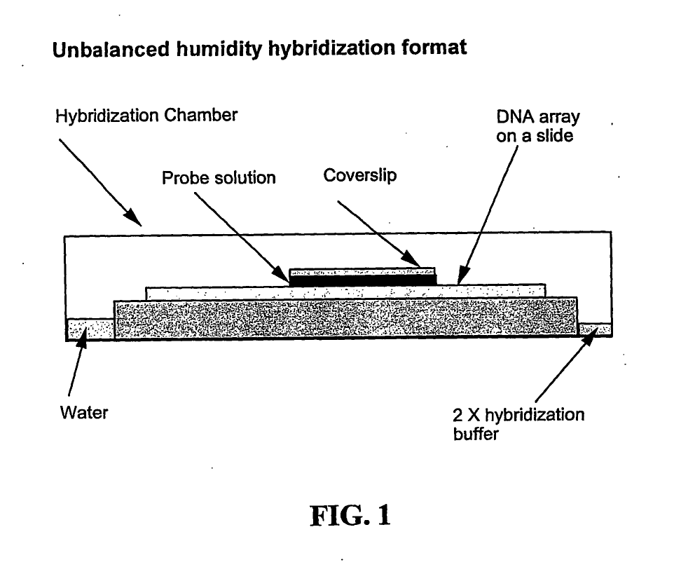

To further increase the hybridization sensitivity, hybridization can be carried out in a controlled, unsaturated humidity environment; thus, hybridization efficiency is significantly improved if the humidity is not saturated. See co-pending U.S. patent application Ser. No. 09/839,658, filed Apr. 19, 2001. The hybridization efficiency can be improved if the humidity is dynamically controlled, i.e., if the humidity changes during hybridization. Mass transfer will be facilitated in a dynamically balanced humidity environment. The humidity in the hybridization environment can be adjusted stepwise or continuously. Array devices comprising housings and controls that allow the operator to control the humidity during pre-hybridization, hybridization, wash and/or detection stages can be used. The device can have detection, control and memory components to allow pre-programming of the humidity (and temperature (see below), and other parameters) during the entire procedural cycle, including pre-hybridization, hybridization, wash and detection steps. See co-pending U.S. patent application Ser. No. 09/839,658, filed Apr. 19, 2001. FIG. 1 is a schematic illustration of an exemplary humidified hybridization chamber and an unbalanced humidity hybridization format of the invention.

The methods of the invention can incorporate hybridization conditions comprising temperature fluctuation. Hybridization has much better efficiency in a changing temperature environment as compared to conditions where the temperature is set precisely or at relatively constant level (e.g., plus or minus a couple of degrees, as with most commercial ovens). Reaction chamber temperatures can be fluctuatingly modified by, e.g., an oven, or other device capable of creating changing temperatures. See co-pending U.S. patent application Ser. No. 09/839,658, filed Apr. 19, 2001.

The methods of the invention can comprise hybridization conditions comprising osmotic fluctuation. Hybridization efficiency (i.e., time to equilibrium) can also be enhanced by a hybridization environment that comprises changing hyper-/hypo-tonicity, e.g., a solute gradient. A solute gradient is created in the device. For example, a low salt hybridization solution is placed on one side of the array hybridization chamber and a higher salt buffer is placed on the other side to generate a solute gradient in the chamber. See co-pending U.S. patent application Ser. No. 09/839,658, filed Apr. 19, 2001.

Polypeptides

The invention is also directed to arrays comprising labeled immobilized polypeptides, peptides and peptidomimetics. The polypeptides, peptides and peptidomimetics can be immobilized to a substrate surface using any methodology, including covalent or non-covalent, direct or indirect, attachment to a surface.

For example, a polypeptide can be modified by reaction with a compound having the formula: R₁—X—R₂, where R₁ is a cyclic ether group or an amino group, R₂ is an alkoxysilane group and X is a moiety chemically suitable for linking the cyclic ether group or the amino group to the alkoxysilane group. As noted above, the terms “polypeptide,” “protein,” and “peptide,” used to practice the invention, include compositions of the invention that also include “analogs,” or “conservative variants” and “mimetics” or “peptidomimetics.” The terms “mimetic” and “peptidomimetic” refer to a synthetic chemical compounds. The mimetic can be either entirely composed of synthetic, non-natural analogues of amino acids, or, is a chimeric molecule of partly natural peptide amino acids and partly non-natural analogs of amino acids. The mimetic can also incorporate any amount of natural amino acid conservative substitutions as long as such substitutions also do not substantially alter the mimetics' structure and/or activity. Polypeptide mimetic compositions can contain any combination of non-natural structural components, which are typically from three structural groups: a) residue linkage groups other than the natural amide bond (“peptide bond”) linkages; b) non-natural residues in place of naturally occurring amino acid residues; or c) residues which induce secondary structural mimicry, i.e., to induce or stabilize a secondary structure, e.g., a beta turn, gamma turn, beta sheet, alpha helix conformation, and the like. A polypeptide can be characterized as a mimetic when all or some of its residues are joined by chemical means other than natural peptide bonds. Individual peptidomimetic residues can be joined by peptide bonds, other chemical bonds or coupling means, such as, e.g., glutaraldehyde, N-hydroxysuccinimide esters, bifunctional maleimides, N,N′-dicyclohexylcarbodiimide (DCC) or N,N′-diisopropylcarbodiimide (DIC). Linking groups that can be an alternative to the traditional amide bond (“peptide bond”) linkages include, e.g., ketomethylene (e.g., —C(═O)—CH₂— for —C(═O)—NH—), aminomethylene (CH₂—NH), ethylene, olefin (CH═CH), ether (CH₂—O), thioether (CH₂—S), tetrazole (CN₄—), thiazole, retroamide, thioamide, or ester (see, e.g., Spatola (1983) in Chemistry and Biochemistry of Amino Acids, Peptides and Proteins, Vol. 7, pp 267-357, “Peptide Backbone Modifications,” Marcell Dekker, N.Y.). A polypeptide can also be characterized as a mimetic by containing all or some non-natural residues in place of naturally occurring amino acid residues; non-natural residues are well described in the scientific and patent literature. The skilled artisan will recognize that individual synthetic residues and polypeptides incorporating mimetics can be synthesized using a variety of procedures and methodologies, which are well described in the scientific and patent literature, e.g., Organic Syntheses Collective Volumes, Gilman, et al., supra Polypeptides incorporating mimetics can also be made using solid phase synthetic procedures, as described, e.g., by U.S. Pat. No. 5,422,426. Peptides and peptide mimetics can also be synthesized using combinatorial methodologies. Various techniques for generation of peptide and peptidomimetic libraries are well known, and include, e.g., multipin, tea bag, and split-couple-mix techniques; see, e.g., al-Obeidi (1998) Mol. Biotechnol. 9:205-223; Hruby (1997) Curr. Opin. Chem. Biol. 1:114-119; Ostergaard (1997) Mol. Divers. 3:17-27; Ostresh (1996) Methods Enzymol. 267:220-234. Modified polypeptide and peptides can be further produced by chemical modification methods, see, e.g., Belousov (1997) Nucleic Acids Res. 25:3440-3444; Frenkel (1995) Free Radic. Biol. Med. 19:373-380; Blommers (1994) Biochemistry 33:7886-7896. These peptides can also be synthesized, whole or in part, using chemical methods well known in the art (see e.g., Caruthers (1980) Nucleic Acids Res. Symp. Ser. 215-223; Horn (1980) Nucleic Acids Res. Symp. Ser. 225-232; Banga, A. K., Therapeutic Peptides and Proteins, Formulation, Processing and Delivery Systems (1995) Technomic Publishing Co., Lancaster, Pa. Peptide synthesis can be performed using various solid-phase techniques (see e.g., Roberge (1995) Science 269:202; Merrifield (1997) Methods Enzymol. 289:3-13) and automated synthesis may be used. See also, U.S. Pat. Nos. 6,245,886; 6,169,073; 6,034,211.

Arrays, or “BioChips”

The invention provides improved variations of “arrays” or “microarrays” or “DNA arrays” or “nucleic acid arrays” or “biochips.” The present invention can be practiced with any known array, or variation thereof. Arrays are generically a plurality of “target elements,” each target element comprising a defined amount of one or more biological molecules, e.g., polypeptides, nucleic acid molecules, or probes, immobilized a solid surface for specific binding, e.g., hybridization, to sample molecules. Immobilized nucleic acids can contain sequences from specific messages (e.g., as cDNA libraries) or genes (e.g., genomic libraries), including, e.g., substantially all or a subsection of a chromosome or substantially all of a genome, including a human genome. Other target elements can contain reference sequences and the like. The target elements of the arrays may be arranged on the solid surface at different sizes and different densities. The target element densities will depend upon a number of factors, such as the nature of the label, the solid support, and the like. Each target element may comprise substantially the same nucleic acid sequences, or, a mixture of nucleic acids of different lengths and/or sequences. Thus, for example, a target element may contain more than one copy of a cloned piece of DNA, and each copy may be broken into fragments of different lengths, as described herein. The length and complexity of the nucleic acid fixed onto the array surface is not critical to the invention. The array can comprise nucleic acids immobilized on a solid surface (e.g., nitrocellulose, glass, quartz, fused silica, plastics and the like). See, e.g., U.S. Pat. No. 6,063,338 describing multi-well platforms comprising cycloolefin polymers if fluorescence is to be measured.

In making and using the arrays and methods of the invention, known arrays and methods of making and using arrays can be incorporated in whole or in part, or variations thereof, as described, for example, in U.S. Pat. Nos. 6,277,628; 6,277,489; 6,261,776; 6,258,606; 6,054,270; 6,048,695; 6,045,996; 6,022,963; 6,013,440; 5,965,452; 5,959,098; 5,856,174; 5,830,645; 5,770,456; 5,632,957; 5,556,752; 5,143,854; 5,807,522; 5,800,992; 5,744,305; 5,700,637; 5,556,752; 5,434,049; see also, e.g., WO 99/51773; WO 99/09217; WO 97/46313; WO 96/17958; see also, e.g., Johnston (1998) Curr. Biol. 8:R171-R174; Schummer (1997) Biotechniques 23:1087-1092; Kern (1997) Biotechniques 23:120-124; Solinas-Toldo (1997) Genes, Chromosomes & Cancer 20:399-407; Bowtell (1999) Nature Genetics Supp. 21:25-32. The present invention can be used to modify any known array, e.g., GeneChips™, Affymetrix, Santa Clara, Calif.; SpectralChip™ Mouse BAC Arrays, SpectralChip™ Human BAC Arrays and Custom Arrays of Spectral Genomics, Houston, Tex. The arrays of the invention can comprise housing comprising components for controlling humidity and temperature during the hybridization and wash reactions.

Substrate Surfaces

The arrays of the invention can have substrate surfaces of a rigid, semi-rigid or flexible material. The substrate surface can be flat or planar, be shaped as wells, raised regions, etched trenches, pores, beads, filaments, or the like. Substrates can be of any material upon which a “capture probe” can be directly or indirectly bound. For example, suitable materials can include paper, glass (see, e.g., U.S. Pat. No. 5,843,767), ceramics, quartz or other crystalline substrates (e.g. gallium arsenide), metals, metalloids, polacryloylmorpholide, various plastics and plastic copolymers, Nylon™, Teflon™, polyethylene, polypropylene, poly(4-methylbutene), polystyrene, polystyrene/latex, polymethacrylate, poly(ethylene terephthalate), rayon, nylon, poly(vinyl butyrate), polyvinylidene difluoride (PVDF) (see, e.g., U.S. Pat. No. 6,024,872), silicones (see, e.g., U.S. Pat. No. 6,096,817), polyformaldehyde (see, e.g., U.S. Pat. Nos. 4,355,153; 4,652,613), cellulose (see, e.g., U.S. Pat. No. 5,068,269), cellulose acetate (see, e.g., U.S. Pat. No. 6,048,457), nitrocellulose, various membranes and gels (e.g., silica aerogels, see, e.g., U.S. Pat. No. 5,795,557), paramagnetic or superparamagnetic microparticles (see, e.g., U.S. Pat. No. 5,939,261) and the like. Reactive functional groups can be, e.g., hydroxyl, carboxyl, amino groups or the like. Silane (e.g., mono- and dihydroxyalkylsilanes, aminoalkyltrialkoxysilanes, 3-aminopropyl-triethoxysilane, 3-aminopropyltrimethoxysilane) can provide a hydroxyl functional group for reaction with an amine functional group.

Detectable Labels and Labeling of Biological Molecules

The methods and compositions of the invention use biological molecules, e.g., nucleic acids, that are associated with a detectable label, e.g., have incorporated or have been conjugated to a detectable moiety. The association with the detectable moiety can be covalent or non-covalent. In another aspect, the array-immobilized nucleic acids and test sample nucleic acid are differentially detectable, e.g., they emit difference signals.

Useful labels include, e.g., ³²P, 35S, ³H, ¹⁴C, ¹²⁵I, ¹³¹I; fluorescent dyes (e.g., Cy5™, Cy3™, FITC, rhodamine, lanthanide phosphors, Texas red), electron-dense reagents (e.g. gold), enzymes, e.g., as commonly used in an ELISA (e.g., horseradish peroxidase, beta-galactosidase, luciferase, alkaline phosphatase), colorimetric labels (e.g. colloidal gold), magnetic labels (e.g. Dynabeads™), biotin, dioxigenin, or haptens and proteins for which antisera or monoclonal antibodies are available. The label can be directly incorporated into the nucleic acid or other target compound to be detected, or it can be attached to a probe or antibody that hybridizes or binds to the target. A peptide can be made detectable by incorporating (e.g., into a nucleoside base) predetermined polypeptide epitopes recognized by a secondary reporter (e.g., leucine zipper pair sequences, binding sites for secondary antibodies, transcriptional activator polypeptide, metal binding domains, epitope tags). Label can be attached by spacer arms of various lengths to reduce potential steric hindrance or impact on other useful or desired properties. See, e.g., Mansfield (1995) Mol Cell Probes 9:145-156. In array-based CGH, typically fluors are paired together (one labeling control and another the test nucleic acid), e.g., rhodamine and fluorescein (see, e.g., DeRisi (1996) Nature Genetics 14:458-460), or lissamine-conjugated nucleic acid analogs and fluorescein-conjugated nucleotide analogs (see, e.g., Shalon (1996) supra); or Spectrum Red™ and Spectrum Green™ (Vysis, Downers Grove, Ill.) or Cy3™ and Cy5™ (see below).

Cyanine and related dyes, such as merocyanine, styryl and oxonol dyes, are particularly strongly light-absorbing and highly luminescent, see, e.g., U.S. Pat. Nos. 4,337,063; 4,404,289; 6,048,982. Cy3™ and Cy5™ can be used together; both are fluorescent cyanine dyes produced by Amersham Life Sciences (Arlington Heights, Ill.).

Detectable moieties can be incorporated into array-immobilized nucleic acid and, if desired, “target” nucleic acid, by transcription (e.g., by random-primer labeling using Klenow polymerase, or “nick translation,” or, amplification, or equivalent). For example, in one aspect, a nucleoside base is conjugated to a detectable moiety, such as a fluorescent dye, e.g., Cy3™ or Cy5™, and then incorporated into a nucleic acid for immobilization onto an array or for use as a sample nucleic acid. Samples of genomic DNA can be incorporated with Cy3™- or Cy5™-dCTP conjugates mixed with unlabeled dCTP. According to manufacturer's instructions, if generating labeled target by PCR, a mixture of 33% modified to 66% unmodified dCTP gives maximal incorporation of label; when modified dCTP made up 50% or greater, the PCR reaction was inhibited. Cy5™ is typically excited by the 633 nm line of HeNe laser, and emission is collected at 680 nm. See also, e.g., Bartosiewicz (2000) Archives of Biochem. Biophysics 376:66-73; Schena (1996) Proc. Natl. Acad. Sci. USA 93:10614-10619; Pinkel (1998) Nature Genetics 20:207-211; Pollack (1999) Nature Genetics 23:41-46.

In another aspect, when using PCR or nick translation to label nucleic acids, modified nucleotides synthesized by coupling allylamine-dUTP to the succinimidyl-ester derivatives of the fluorescent dyes or haptenes (such as biotin or digoxigenin) are used; this method allows custom preparation of most common fluorescent nucleotides, see, e.g., Henegariu (2000) Nat. Biotechnol. 18:345-348. Other fluorescent nucleotide analogs can be used, see, e.g., Jameson (1997) Methods Enzymol. 278:363-390; Zhu (1994) Nucleic Acids Res. 22:3418-3422. U.S. Pat. Nos. 5,652,099 and 6,268,132 also describe nucleoside analogs for incorporation into nucleic acids, e.g., DNA and/or RNA, or oligonucleotides, via either enzymatic or chemical synthesis to produce fluorescent oligonucleotides. U.S. Pat. No. 5,135,717 describes phthalocyanine and tetrabenztriazaporphyrin reagents for use as fluorescent labels.

In the compositions and methods of the invention, labeling with a detectable composition (labeling with a detectable moiety) also can include a nucleic acid attached to another biological molecule, such as a nucleic acid, e.g., a nucleic acid in the form of a stem-loop structure as a “molecular beacon” or an “aptamer beacon.” Molecular beacons as detectable moieties are well known in the art; for example, Sokol (1998) Proc. Natl. Acad. Sci. USA 95:11538-11543, synthesized “molecular beacon” reporter oligodeoxynucleotides with matched fluorescent donor and acceptor chromophores on their 5′ and 3′ ends. In the absence of a complementary nucleic acid strand, the molecular beacon remains in a stem-loop conformation where fluorescence resonance energy transfer prevents signal emission. On hybridization with a complementary sequence, the stem-loop structure opens increasing the physical distance between the donor and acceptor moieties thereby reducing fluorescence resonance energy transfer and allowing a detectable signal to be emitted when the beacon is excited by light of the appropriate wavelength. See also, e.g., Antony (2001) Biochemistry 40:9387-9395, describing a molecular beacon comprised of a G-rich 18-mer triplex forming oligodeoxyribonucleotide. See also U.S. Pat. Nos. 6,277,581 and 6,235,504.

Aptamer beacons are similar to molecular beacons; see, e.g., Hamaguchi (2001) Anal. Biochem. 294:126-131; Poddar (2001) Mol. Cell. Probes 15:161-167; Kaboev (2000) Nucleic Acids Res. 28:E94. Aptamer beacons can adopt two or more conformations, one of which allows ligand binding. A fluorescence-quenching pair is used to report changes in conformation induced by ligand binding. See also, e.g., Yamamoto (2000) Genes Cells 5:389-396; Smirnov (2000) Biochemistry 39:1462-1468.

In addition to methods for labeling nucleic acids with fluorescent dyes, methods for the simultaneous detection of multiple fluorophores are well known in the art, see, e.g., U.S. Pat. Nos. 5,539,517; 6,049,380; 6,054,279; 6,055,325. For example a spectrograph can image an emission spectrum onto a two-dimensional array of light detectors; a full spectrally resolved image of the array is thus obtained. Photophysics of the fluorophore, e.g., fluorescence quantum yield and photodestruction yield, and the sensitivity of the detector are read time parameters for an oligonucleotide array. With sufficient laser power and use of Cy5™ and/or Cy3™, which have lower photodestruction yields an array can be read in less than 5 seconds.

When using two or more fluors together (e.g., as in a CGH), such as Cy3™ and Cy5™, it is necessary to create a composite image of all the fluors. To acquire the two or more images, the array can be scanned either simultaneously or sequentially. Charge-coupled devices, or CCDs, are used in microarray scanning systems, including the multiplexed systems of the invention. Thus, CCDs used in the systems and methods of the invention can scan and analyze multicolor fluorescence images; see, e.g., U.S. Pat. Nos. 6,261,776; 6,252,664; 6,191,425; 6,143,495; 6,140,044; 6,066,459; 5,943,129; 5,922,617; 5,880,473; 5,846,708; 5,790,727; and, the patents cited in the discussion of arrays, herein.

The methods of the invention further comprise data analysis, which can include the steps of determining, e.g., fluorescent intensity as a function of substrate position, removing “outliers” (data deviating from a predetermined statistical distribution), or calculating the relative binding affinity of the targets from the remaining data. The resulting data can be displayed as an image with color in each region varying according to the light emission or binding affinity between targets and probes. See, e.g., U.S. Pat. Nos. 5,324,633; 5,863,504; 6,045,996. The invention can also incorporate a device for detecting a labeled marker on a sample located on a support, see, e.g., U.S. Pat. No. 5,578,832.

Fragmentation and Digestion of Nucleic Acid

In practicing the methods and compositions of the invention, immobilized and sample nucleic acids can be in a variety of lengths. For example, in one aspect, the biological molecule analyzed is derived from a genomic nucleic acid, and, labeled sample fragments consist of a length smaller than about 200 bases. Use of labeled genomic DNA limited to this small size significantly improves the resolution of the molecular profile analysis, e.g., in array-based CGH. For example, use of such small fragments allows for significant suppression of repetitive sequences and other unwanted, “background” cross-hybridization on the immobilized nucleic acid. Suppression of repetitive sequence hybridization greatly increases the reliability of the detection of copy number differences (e.g., amplifications or deletions) or detection of unique sequences. See co-pending U.S. patent application Ser. No. 09/839,658, filed Apr. 19, 2001.

The resultant fragment lengths can be modified by, e.g., treatment with DNase. Adjusting the ratio of DNase to DNA polymerase in a nick translation reaction changes the length of the digestion product. Standard nick translation kits typically generate 300 to 600 base pair fragments. If desired, the labeled nucleic acid can be further fragmented to segments below 200 bases, down to as low as about 25 to 30 bases, random enzymatic digestion of the DNA is carried out, using, e.g., a DNA endonucleases, e.g., DNase (see, e.g., Herrera (1994) J. Mol. Biol. 236:405-411; Suck (1994) J. Mol. Recognit. 7:65-70), or, the two-base restriction endonuclease CviJI (see, e.g., Fitzgerald (1992) Nucleic Acids Res. 20:3753-3762) and standard protocols, see, e.g., Sambrook, Ausubel, with or without other fragmentation procedures.

Other procedures can also be used to fragment genomic DNA, e.g. mechanical shearing, sonication (see, e.g., Deininger (1983) Anal. Biochem. 129:216-223), and the like (see, e.g., Sambrook, Ausubel, Tijssen). For example, one mechanical technique is based on point-sink hydrodynamics that result when a DNA sample is forced through a small hole by a syringe pump, see, e.g., Thorstenson (1998) Genome Res. 8:848-855. See also, Oefner (1996) Nucleic Acids Res. 24:3879-3886; Ordahl (1976) Nucleic Acids Res. 3:2985-2999. Fragment size can be evaluated by a variety of techniques, including, e.g., sizing electrophoresis, as by Siles (1997) J. Chromatogr. A. 771:319-329, that analyzed DNA fragmentation using a dynamic size-sieving polymer solution in a capillary electrophoresis. Fragment sizes can also be determined by, e.g., matrix-assisted laser desorption/ionization time-of-flight mass spectrometry, see, e.g., Chiu (2000) Nucleic Acids Res. 28:E31.

Generating Molecular Profiles of Sample Nucleic Acids

The invention provides compositions and methods for generating molecular profiles of biological samples, e.g., nucleic acid samples, such as samples of genomic DNA or a cDNA library. In one aspect, using the arrays and practicing the methods of the invention, array-bound nucleic acids are contacted with a sample comprising nucleic acids; the binding of the sample nucleic acids to the array is detected to generate a molecular profile of the sample nucleic acid. In alternative aspects, the molecular profile can be a comparative genomic hybridization (CGH) reaction; detection of a genomic DNA amplification, a genomic DNA deletion, or a genomic DNA insertion; detection of a point mutation, such as identification of a single-nucleotide polymorphism (SNP); differential methylation hybridization (DMH), where the array-bound nucleic acids are CpG island tags; detection of transcriptionally active regions of a genome (using, e.g., nuclear run-off assays); analysis of a chromatin structure; and analysis of a telomeric structure (such as telomeric erosion or telomeric addition). All of these procedures are well known in the art, and any molecular biology procedure or analysis, can be performed using the modified biological molecules or arrays of the invention. See also, co-pending U.S. patent application Ser. No. 09/853,343.

Comparative Genomic Hybridization (CGH)

The arrays and methods of the invention are used in comparative genomic hybridization (CGH) reactions. Thus, in one aspect, labeled genomic nucleic acid is immobilized onto substrate surfaces. CGH is a molecular cytogenetics approach that can be used to detect regions in a genome undergoing quantitative changes, i.e. gains or losses of copy numbers. Analysis of genomes of tumor cells can detect a region or regions of anomaly under going gains and/or losses. Differential expression of hundreds of genes can be analyzed using a cDNA array, thus facilitating characterization of gene expression in normal and diseased tissues. Generating a molecular profile of a nucleic acid sample by comparative genomic hybridization using methods and arrays of the invention can be practiced with methods and compositions known in the art, see, e.g., U.S. Pat. Nos. 6,197,501; 6,159,685; 5,976,790; 5,965,362; 5,856,097; 5,830,645; 5,721,098; 5,665,549; 5,635,351; and, Diago (2001) American J. of Pathol. May;158(5):1623-1631; Theillet (2001) Bull. Cancer 88:261-268; Werner (2001) Pharmacogenomics 2:25-36; Jain (2000) Pharmacogenomics 1:289-307.

Detection of Single-Nucleotide Polymorphisms (SNPs)

In one aspect, the arrays and methods of the invention are used to detect point mutations, such as single-nucleotide polymorphisms (SNPs). Thus, in one aspect, labeled nucleic acid for detecting SNPs is immobilized onto substrate surfaces. Arrays can be used for high-throughput genotyping approaches for pharmacogenomics, where numerous individuals are studied with thousands of SNP markers. Generating a molecular profile of a nucleic acid sample by the analysis and detection of SNPs using methods and arrays of the invention can be practiced with methods and compositions known in the art, see, e.g., U.S. Pat. Nos. 6,221,592; 6,110,709; 6,074,831; 6,015,888; and, Kwok (2000) Pharmacogenomics 1:95-100; Riley (2000) Pharmacogenomics 1:39-47; Kokoris (2000) Mol. Diagn. 5:329-340; Shi (2001) Clin. Chem. 47:164-172; Fan (2000) Genome Res. 10:853-860; Ianonne (2000) Cytometry 39:131-140; Cai (2000) Genomics.66:135-143; Chen (2000) Genome Res. 10:549-557; Syvanen (1999) Hum. Mutat. 13:1-10; Pastinen (1997) Genome Res. 7:606-614.

Differential Methylation Hybridization (DMH)

The arrays and methods of the invention are used in differential methylation hybridization (DMH), including, for example, CpG island analysis. Thus, in one aspect, the array-bound labeled nucleic acids comprise CpG island tags. In one aspect, the methods and arrays of the invention are used to identify, analyze and map hypermethylated or hypomethylated regions of the genome. In one aspect, the sample nucleic acids can comprise genomic DNA digested with at least one methylation-sensitive restriction endonuclease and the molecular profile comprises detection and mapping of hypermethylated (or hypomethylated) regions of the genome. Any methylation-sensitive restriction endonuclease or equivalent endonuclease enzyme can be used, including, for example, NotI, SmaI, SacII, EagI, MspI, HpaII, Sau3AI and BssHII. In one aspect of the methods of the invention, both a methylation-sensitive enzyme and its methylation insensitive isoschizomer is used; see, e.g., Robinson (2000) Chromosome Res. 8:635-643; described use of the methylation-sensitive enzyme HpaII and its methylation insensitive isoschizomer MspI. Windhofer (2000) Curr. Genet. 37:194-199, described digestion of genomic DNA with the methylation-sensitive endonuclease Sau3AI and the methylation-insensitive endonuclease NdeII. See also, e.g., Muller (2001) J. Biol. Chem. 276:14271-14278; Memisoglu (2000) J. Bacteriol. 182:2104-2112; Roth (2000) Biol. Chem. 381:269-272. Generating a molecular profile of a nucleic acid sample by the analysis of differential methylation and CpG islands using methods and arrays of the invention can be practiced with methods and compositions known in the art, see, also, U.S. Pat. Nos. 6,214,556; 6,180,344; 5,851,762; and, WO0127317, WO9928498; WO0044934; and WO1999DE03747 19991119.

Analysis of Telomeric Structure

The arrays and methods of the invention are used in the analysis of a telomeric structure, such as telomeric erosion or telomeric addition. Thus, in one aspect, labeled nucleic acid comprising telomeric structures, or, labeled telomeric structures alone, are immobilized onto substrate surfaces. Telomerase assays are useful for cancer detection and diagnosis (see, e.g., Hahn (2001) Ann Med 33:123-129; Meyerson (2000) J. Clin. Oncol. 18:2626-2634; Meyerson (1998) Toxicol. Lett. 102-103:41-5). Using the array-based telomeric structures of the invention will accelerate understanding of telomerase biology and lead to clinically relevant telomerase-based therapies. Generating a molecular profile of a nucleic acid sample by the analysis of telomeric structures using methods and arrays of the invention can be practiced with methods and compositions known in the art, see, e.g., U.S. Pat. Nos. 6,221,590; 6,221,584; 6,022,709; 6,007,989; 6,004,939; 5,972,605; 5,871,926; 5,834,193; 5,830,644; 5,695,932; 5,645,986.

Analysis of Chromatin Structure

The arrays and methods of the invention are used in the analysis of chromatin structure, including chromatin condensation, chromatin decondensation, histone phosphorylation, histone acylation, and the like (see, e.g., Guo (2000) Cancer Res. 60:5667-5672; Mahlknecht (2000) Mol. Med. 6:623-644). Thus, in one aspect, labeled nucleic acid comprising chromatin structures, or, labeled chromatin structures alone, are immobilized onto substrate surfaces. Chromatin structure remodeling occurs in certain cancers (see, e.g., Giamarchi (2000) Adv. Exp. Med. Biol. 480:155-161). Chromatin structure affects nuclear processes that utilize DNA as a substrate, e.g., transcription, replication, DNA repair, and DNA organization within the nucleus. Chromatin structure analysis is useful in fertility assessment; for example, sperm with decondensed chromatin are infertile. DNA damage in patients with untreated cancer can be measured using a sperm chromatin structure assay (see, e.g., Kobayashi (2001) Fertil. Steril. 75:469-475). Generating a molecular profile of a nucleic acid sample by the analysis of chromatin structure using the methods and arrays of the invention can be practiced with methods and compositions known in the art, see, e.g., U.S. Pat. Nos. 6,204,064; 6,187,749; 6,097,485; 5,972,608; 5,919,621; 5,470,709; and, Dreyer (2000) Anal. Cell Pathol. 20:141-150; Hong (2001) Acta Cytol. 45:163-168; Evenson (1991) Reprod. Toxicol. 5:115-125.

It is understood that the examples and embodiments described herein are for illustrative purposes only and that various modifications or changes in light thereof will be suggested to persons skilled in the art and are to be included within the spirit and purview of this application and scope of the appended claims.

### EXAMPLES

The following example is offered to illustrate, but not to limit the claimed invention.

### Example 1

### Making Nucleic Acid Arrays

The following example demonstrates exemplary protocol for making an array of the invention.

Making BAC Microarrays:

BAC clones greater than fifty kilobases (50 kb), and up to about 300 kb, are grown up in Terrific Broth medium. Larger inserts, e.g., clones >300 kb, and smaller inserts, about 1 to 20 kb, are also be used. DNA is prepared by a modified alkaline lysis protocol (see, e.g., Sambrook). The DNA is labeled, as described below.

The DNA is then chemically modified as described by U.S. Pat. No. 6,048,695. The modified DNA is then dissolved in proper buffer and printed directly on clean glass surfaces as described by U.S. Pat. No. 6,048,695. Usually multiple spots are printed for each clone.

Nucleic Acid Labeling and DNase Enzyme Fragmentation:

A standard random priming method is used to label genomic DNA before its attachment to the array, see, e.g., Sambrook. Sample nucleic acid is also similarly labeled. Cy3™ or Cy5™ labeled nucleotides are supplemented together with corresponding unlabeled nucleotides at a molar ratio ranging from 0.0 to about 6 (unlabeled nucleotide to labeled nucleotides). Labeling is carried out at 37° C. for 2 to 10 hours. After labeling the reaction mix is heated up to 95° C. to 100° C. for 3 to 5 minutes to inactivate the polymerase and denature the newly generated, labeled “probe” nucleic acid from the template.

The heated sample is then chilled on ice for 5 minutes. “Calibrated” DNase (DNA endonuclease) enzyme is added to fragment the labeled template (generated by random priming). “Trace” amounts of DNase is added (final concentration was 0.2 to 2 ng/ml; incubation time 15 to 30 minutes) to digest/fragment the labeled nucleic acid to segments of about 30 to about 100 bases in size.

A number of embodiments of the invention have been described. Nevertheless, it will be understood that various modifications may be made without departing from the spirit and scope of the invention. Accordingly, other embodiments are within the scope of the following claims.

1. An array comprising a plurality of biological molecules, wherein each biological molecule is immobilized to a discrete and known spot on a substrate surface to form an array of biological molecules, and the biological molecule comprises a detectable label.

2. The array of claim 1, wherein the plurality of biological molecules comprises a plurality of nucleic acids.

3. The array of claim 2, wherein the nucleic acid is a DNA or an RNA.

4. The array of claim 2, wherein the nucleic acid is derived from a human.

5. The array of claim 2, wherein the plurality of nucleic acids is genomic DNA or is derived from a genomic DNA.

6. The array of claim 2, wherein the plurality of nucleic acids comprises a plurality of cloned nucleic acid segments.

7. The array of claim 6, wherein a cloned nucleic acid segment comprises a cloned genomic nucleic acid segment.

8. The array of claim 7, wherein the cloned genomic nucleic acid segment is free of deletions, as compared to a corresponding wild type genomic nucleic acid segment.

9. The array of claim 7, wherein the cloned genomic nucleic acid segment is free of additions, as compared to a corresponding wild type genomic nucleic acid segment.

10. The array of claim 7, wherein the cloned genomic nucleic acid segment is free of additions and deletions, as compared to a corresponding wild type genomic nucleic acid segment.

11. The array of claim 7, wherein the cloned genomic nucleic acid segment comprises a gene.

12. The array of claim 6, wherein the cloned nucleic acid comprises a cDNA or a DNA sequence corresponding to or complementary to an RNA message.

13. The array of claim 12, wherein the message encodes a polypeptide.

14. The array of claim 7, wherein the spot consists of a cloned nucleic acids segment comprising a single cloned genomic nucleic acid segment.

15. The array of claim 7, wherein the cloned genomic nucleic acid segments in a first spot is non-overlapping in sequence compared to the cloned genomic nucleic acid segments in a second spot.

16. The array of claim 15, wherein the cloned genomic nucleic acid segments in a spot is non-overlapping in sequence compared to the cloned genomic nucleic acid segments of other spots on the array.

17. The array of claim 7, wherein each cloned genomic nucleic acid segment is spotted in duplicate discrete and known spots on the array.

18. The array of claim 7, wherein the plurality of cloned genomic nucleic acid segments comprises a known segment of a genome.

19. The array of claim 18, wherein the plurality of known segments of the genome comprises a chromosome.

20. The array of claim 19, wherein the chromosome comprises a human chromosome.

21. The array of claim 18, wherein the plurality of known segments of the genome comprises a genome.

22. The array of claim 21, wherein the genome comprises a human genome.

23. The array of claim 7, wherein the cloned nucleic acid segment is cloned in a construct comprising an artificial chromosome.

24. The array of claim 23, wherein the artificial chromosome comprises a bacterial artificial chromosome (BAC).

25. The array of claim 23, wherein the artificial chromosome is selected from the group consisting of a human artificial chromosome (HAC), a yeast artificial chromosome (YAC), a transformation-competent artificial chromosome (TAC) and a bacteriophage P1-derived artificial chromosome (PAC).

26. The array of claim 7, wherein the cloned nucleic acid segment is cloned in a construct comprising a vector selected from the group consisting of a cosmid vector, a plasmid vector and a viral vector.

27. The array of claim 7, wherein the cloned nucleic acid segment is between about 50 kilobases to about 500 kilobases in length.

28. The array of claim 27, wherein the cloned nucleic acid segment is between about 100 kilobases to about 400 kilobases in length.

29. The array of claim 28, wherein the cloned nucleic acid segment is about 300 kilobases in length.

30. The array of claim 1, wherein the detectable label comprises a fluorescent label.

31. The array of claim 30, wherein the fluorescent label comprises Cy5™ or equivalent.

32. The array of claim 30, wherein the fluorescent label comprises Cy3™ or equivalent.

33. The array of claim 30, wherein the fluorescent label comprises a rhodamine, a fluorescein or an aryl-substituted 4,4-difluoro-4-bora-3a,4a-diaza-s-indacene dye or equivalents.

34. The array of claim 1, wherein the detectable label comprises a nucleic acid stem-loop structure.

35. The array of claim 34, wherein the nucleic acid stem-loop structure comprises a molecular beacon structure.

36. The array of claim 34, wherein the nucleic acid stem-loop structure comprises an aptamer beacon structure.

37. The array of claim 1, wherein the biological molecule is covalently bound to the detectable label.

38. The array of claim 1, wherein the immobilized biological molecules comprise the same detectable label.

39. The array of claim 1, wherein about 50% of the immobilized biological molecules comprise a detectable label.

40. (canceled)

41. (canceled)

42. The array of claim 39, wherein about 80% of the array-immobilized biological molecules comprise a detectable label.

43. (canceled)

44. (canceled)

45. (canceled)

46. The array of claim 39, wherein about 99% of the array-immobilized biological molecules comprise a detectable label.

47. (canceled)

48. The array of claim 1, wherein the biological molecule is covalently bound to the substrate surface.

49. The array of claim 48, wherein the biological molecule is covalently bound to a compound having the general formula: R₁—X—R₂, wherein R₁ is a cyclic ether, an aldehyde, or a chloromethylphenyl moiety; X is a moiety chemically suitable for linking the R₁ moiety to the R₂ moiety, and the R₂ moiety has the general formula 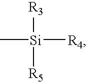
wherein R₃, R₄ and R₅ comprise identical or different alkoxy group or chloro groups.

50. The array of claim 49, wherein the cyclic ether is an epoxide group.

51. The array of claim 50, wherein the epoxide group is an epoxycyclohexyl group or epoxycyclopentyl group.

52. The array of claim 49, wherein the R₂ moiety comprises an alkoxysilane group.

53. The array of claim 49, wherein the R₂ moiety is selected from the group consisting of a methoxyethoxy group, a —OCH₂CH₂OCH₃ group, a chlorohalide group and a propoxy group.

54. The array of claim 49, wherein R₁—X—R₂ compound is selected from the group consisting of (3-glycidoxypropyl) methyldiethoxysilane, (3-glycidoxypropyl)methyldiisopropenoxysilane; 2-(3,4-epoxycyclohexyl) ethyltrimethoxysilane and (3-glycidoxypropyl)dimethylethoxysilane.

55. The array of claim 1, wherein the biological molecule is covalently bound to a compound having the general formula: R₁—X—R₂, wherein R₁ is an amino group, R₂ is an alkoxysilane group or a chlorohalide group; and X is a moiety chemically suitable for linking the R₁ group and the R₂ group.

56. The array of claim 1, wherein the biological molecule is covalently bound to a compound having the general formula
R₁—X—Si(OR₂)ₘ(Cl)ₙ(R)ₖ,
wherein m+k is the integer 3, and n can be 0 if m is greater than 0, or n+k is the integer 3 and m can be 0 if n is greater than 0; X is an inert linker; R₁ comprises a group reactive toward the biological molecule; R is an alkyl group; and, R₂ is an alkyl group.

57. An array comprising a plurality of bacterial artificial chromosomes (BACs), wherein each bacterial artificial chromosome is immobilized to a discrete and known spot on a substrate surface to form an array of bacterial artificial chromosomes (BACs), and each bacterial artificial chromosome comprises a detectable label.

58. The array of claim 57, wherein the array comprises a SpectralChip™ Mouse BAC Array.

59. The array of claim 57, wherein the array comprises a SpectralChip™ Human BAC Array.

60. A method of making an array, wherein the array comprises a plurality of genomic nucleic acids covalently labeled with a compound comprising a fluorescent label, comprising
labeling the plurality of genomic nucleic acid segments, wherein the segments have lengths averaging between about 50 kilobases and about 500 kilobases or are fragmented or digested to have lengths averaging between about 50 kilobases and about 500 kilobases, with a fluorescent label; and
immobilizing the labeled nucleic acids to discrete and known spots on a substrate surface to form an array of nucleic acids.

61. The method of claim 60, wherein a cloned nucleic acid segment is cloned in a construct comprising an artificial chromosome.

62. The method of claim 61, wherein the artificial chromosome comprises a bacterial artificial chromosome (BAC).

63. The method of claim 61, wherein the artificial chromosome is selected from the group consisting of a human artificial chromosome (HAC), a yeast artificial chromosome (YAC), a transformation-competent artificial chromosome (TAC) and a bacteriophage P1-derived artificial chromosome (PAC).

64. The method of claim 60, wherein the detectable label comprises a fluorescent label.

65. The method of claim 64, wherein the fluorescent label comprises Cy5™ or equivalent.

66. The method of claim 64, wherein the fluorescent label comprises Cy3™ or equivalent.

67. The method of claim 64, wherein the fluorescent label comprises a rhodamine, a fluorescein or an aryl-substituted 4,4-difluoro-4-bora-3a,4a-diaza-s-indacene dye or equivalents.

68. The method of claim 60, wherein labeling of the genomic nucleic acid segments comprises labeling by random primer extension.

69. The method of claim 60, wherein labeling of the genomic nucleic acid segments comprises nick translation labeling.

70. A method of performing comparative genomic hybridization (CGH) using an array comprising a plurality of genomic nucleic acids covalently labeled with a compound comprising a detectable label, comprising:
contacting an array comprising a plurality of cloned genomic nucleic acid segments, wherein each genomic nucleic acid segment is immobilized to a discrete and known spot on a substrate surface to form an array, and the genomic nucleic acid segments comprise a first detectable label, with a sample comprising a plurality of genomic nucleic acid segments, wherein the sample genomic nucleic acid segments are labeled with at least a second detectable label; under conditions wherein the nucleic acid in the sample can specifically hybridize to the genomic nucleic acid segments and
measuring an amount of the first and the second fluorescent label on each spot, thereby performing comparative genomic hybridization.

71. The method of claim 70, wherein a cloned nucleic acid segment is cloned in a construct comprising an artificial chromosome.

72. The method of claim 70, wherein the artificial chromosome comprises a bacterial artificial chromosome (BAC).

73. The method of claim 70, wherein the detectable label comprises a fluorescent label.

74. The method of claim 73, wherein the fluorescent label comprises Cy5™ or equivalent.

75. The method of claim 73, wherein the fluorescent label comprises Cy3™ or equivalent.

76. The method of claim 73, wherein the fluorescent label comprises a rhodamine, a fluorescein or an aryl-substituted 4,4-difluoro-4-bora-3a,4a-diaza-s-indacene dye or equivalents.

77. A method of comparing copy numbers of unique nucleic acid sequences in a first sample comprising nucleic acid sequences of a first cell or cell population relative to copy numbers of substantially identical sequences in at least a second sample comprising nucleic acid sequences of at least a second cell or cell population, wherein the nucleic acids of the first sample comprise a first detectable label, and the nucleic acids of the second sample comprise a second detectable label, said method comprising:
contacting an array comprising the nucleic acid sequences of the first sample, wherein the nucleic acid sequences of the first sample are immobilized to discrete and known spots on a substrate surface to form an array, with a second sample comprising nucleic acid sequences of the second sample; under conditions wherein the nucleic acid sequences in the second sample can specifically hybridize to the immobilized nucleic acid sequences of the first sample; and
comparing the intensities of the signals from the nucleic acids sequences of the second sample to the nucleic acid sequences of the second sample, thereby comparing copy numbers of unique nucleic acid sequences in the first sample compared to the second sample.

78. A multiplexed system for performing comparative genomic hybridization (CGH) using an array comprising:
the array comprising a plurality of biological molecules, wherein the biological molecule is immobilized to a discrete and known spot on a substrate surface to form the array of biological molecules, and each biological molecule comprises a first detectable label;
a device for detecting the immobilized first detectable label and at least a second detectable label, wherein the device can measure the first and the second detectable labels on the spots on the substrate surface.

79. The multiplexed system of claim 78, wherein the device comprises a charge-coupled device (CCD).

80. The multiplexed system of claim 78, wherein the device is capable of multicolor fluorescence imaging.

81. The multiplexed system of claim 78, further comprising a computer processor to analyze multicolor fluorescence imaging data.
